(19) United States
(12) Patent Application Publication
(10) Pub. No.: US 2006/0141037 A1
(43) Pub. Date: Jun. 29, 2006

(54) Bilayer tablets of oxcarbazepine for controlled delivery and a process of preparation thereof

(75) Inventors: Bharat Pravinchandra Mehta, Mumbai (IN); Rajen Shah, Maharashtra (IN); Milind Dattatraya Joshi, Maharashtra (IN)

Correspondence Address: LACKENBACH SIEGEL, LLP, LACKENBACH SIEGEL BUILDING, 1 CHASE ROAD, SCARSDALE, NY, 10583, US

(73) Assignee: J. B. Chemicals & Pharmaceuticals Ltd., Worli Bombay (IN)

(21) Appl. No.: 11/314,890

(22) Filed: Dec. 21, 2005

(30) Foreign Application Priority Data
Dec. 29, 2004 (IN) 1425/MUM/2004

(51) Int. Cl. A61K 9/24; A61K 31/55

(52) U.S. Cl. 424/472; 514/217

(57) ABSTRACT

Bilayer tablet comprising an immediate release first layer comprising an effective amount of oxcarbazepine and at least one pharmaceutically acceptable excipients and a controlled release second layer comprising an effective amount of oxcarbazepine and pharmaceutically acceptable excipients wherein the total amount of oxcarbazepine impurities is less than or equal to about 2% by weight. A process for preparation of controlled release bilayer tablets is capable of delivering oxcarbazepine from one layer immediately followed by a controlled delivery of oxcarbazepine from a matrix forming layer, and a process for preparation of oxcarbazepine bilayer tablets. Bilayer tablets of oxcarbazepine, which maintain a therapeutically effective blood concentration of oxcarbazepine with once a day administration.

### BACKGROUND OF THE INVENTION

1. Field of the Invention

This invention generally relates to pharmaceutical compositions for oral administration. This invention relates in particular to such compositions in the form of bilayer tablets including oxcarbazepine for controlled delivery.

The invention relates in particular to Bilayer oxcarbazepine tablets and to a process of preparation of bilayer tablets which offer immediate release of oxcarbazepine followed by controlled release of oxcarbazepine for the treatment of convulsive states.

2. Description of the Prior Art

Oxcarbazepine (10,11-dihydro-10-oxo-5H-dibenz[b,f]azepine-5-carboxamide). It is used as a monotherapy or adjunctive therapy in the treatment of partial seizures with or without secondarily generalized tonic clonic seizures.

Oxcarbazepine and its pharmacologically active metabolite, 10-monohydroxy derivative (MHD; 10,11-dihydro-10-hydro-carbamazepine) show potent antiepileptic activity in animal models comparable to that of carbamazepine and phenytoin. Oxcarbazepine and MHD have been shown to exert antiepileptic activity by blockade of voltage-dependent sodium channels in the brain.

The pharmacological activity of oxcarbazepine is primarily exerted through the 10-monohydroxy metabolite (MHD) of oxcarbazepine. The precise mechanism by which oxcarbazepine and MHD exert their antiseizure effect is unknown; however in vitro electrophysiological studies indicate that they produce a blockade of voltage-sensitive sodium channels, resulting in the stabilization of hyperexcited neural membranes, inhibition of repetitive neuronal firing, and dimunition of propagation of synaptic impulses. These actions are thought to be important in the prevention of seizure spread in the intact brain. In addition, increased potassium conduction and modulation of high-voltage activated calcium channels may contribute to the anticonvulsive effects of the drug. Oxcarbazepine is oxidatively metabolised by the liver to produce pharmacologically active 10-hydroxycarbamazepine. Oxcarbazepine is used in the treatment of partial seizures.

U.S. Pat. Nos. 5,472,714 and 5,695,782 and PCT application WO 98/35681 (TRILEPTAL®. Ciba-Geigy) describe colour stable double-layered Oxcarbazepine tablets. The colour stability is achieved by providing a double coating to the tablets. Oxcarbazepine tablets are known to develop pale orange colour during storage. The colour change is due to the formation of a minor amount of pharmacologically harmless oxidation product to ensure an external homogeneity of product iron (II) oxide which is added. The double-layered tablets described comprise of a tablet core containing drug, a hydrophilic permeable inner layer containing titanium dioxide pigments and a hydrophilic permeable outer layer containing titanium dioxide pigments in combination with iron (II) oxide pigments. The tablet cores are manufactured by conventional wet granulation or direct compression method and subsequently coated with an inner film layer and outer layer containing iron oxide pigment. These tablets are immediate release tablets.

Oxcarbazepine dosage form is described in US published patent application number US 2004/0197402 A1 and PCT application number PCT/TB02/01720 (Ranbaxy Inc.). The application discloses a dosage form for oral administration comprising of oxcarbazepine and a wetting agent. The tablets are designed to improve dissolution rate or bioavailability of oxcarbazepine. These are conventional tablets and provide immediate release of oxcarbazepine.

The concept of a bilayer tablet is well known in the art which is generally employed for various purposes such as stabilization (U.S. Pat. No. 6,287,600), taste masking (U.S. Pat. No. 5,690,959) or delivering two drugs having synergistic effects (U.S. Pat. No. 6,319,519). Bastin described use of bilayer tablet for administration of drugs prone to abuse where the drug layer and gelling layer are separate and the concentration of gelling agent is such that it does not retard release of active agent but facilitates drug release similar to that of a conventional tablet. Blume (U.S. Pat. No. 6,372,252) discloses guaifenesin sustained release bilayer tablet offering bioavailability of drug for 12 hours where first portion is of immediate release and second is for controlled release. However in this invention the bioavailability is only for 12 hours.

Bilayer tablets described in present invention comprise two layers, one layer containing a drug for immediate release and other layer containing a drug for controlled release. The present invention is designed for oxcarbazepine and provides bioavailability for 24 hours.

For controlled release of drug osmotic dosage forms have been described. U.S. Pat. No. 5,192,550 (OROS.®., Alza Corporation) describes one such osmotic dosage form for treating central nervous system disorders. It consists of a compartment, a wall surrounding that compartment and an exit in the wall that connects the exterior with the interior of the dosage form for delivery of drug. The compartment comprises a drug layer and an osmotically effective push layer. The compartment is surrounded with a wall containing a drug for immediate release and aqueous film forming polymers. There is an exit in the wall-connecting compartment to external environment. Water permeates from surrounding body fluids through the semi permeable wall and the pressure that is built up causes a solution/suspension of the drug to be released from the passageway at controlled rate.

Another oral osmotic controlled drug delivery system for slightly soluble drugs is described in U.S. Pat. No. 6,534,090. This osmotic system comprises a core containing carbamazepine, water soluble polymers for inducing osmosis, a crystal habit modifier. It is an advancement over U.S. Pat. No. 5,192,550 as it employs a crystal habit modifier in whose presence upon contact with water, the anhydrous carbamazepine converts to cuboidal, or rod-shaped crystals, or mixtures thereof.

Designs of osmotic delivery systems usually consist of a compartment surrounded by a wall and an orifice in the wall connecting compartment to exterior environment. Preparation of these osmotic devices is complicated e.g. formation of the orifice requires laser drilling which is advanced technology and expensive.

Formation of a wall around the compartment often uses coating technology, and organic solvents are used in coating. Moreover, the amount of drug added is more than the dose of the drug in order to achieve a constant release. This may lead to an increase in cost of drugs.

Therefore, there has not been a fully satisfactory and economical formulation for providing a predictable and uniform treatment regimen, which avoids the need for the construction of complex devices for oral administration and that have the further advantage of simplifying treatment and improving patient compliance and prolonging the release of the drug.

It shall be appreciated that a bilayer tablet of oxcarbazepine providing an immediate release followed by controlled release is novel, inventive and an improvement in drug release tablets.

The fact that the dosage form of the present invention is manufactured without the use of organic solvents makes them safer for patients and for operators who manufacture and handle them. The dosage form disclosed is preferably uncoated and if coated it uses aqueous solvents for coating and thus safer for patients, because there is no need to consider residual organic impurities associated with organic solvents which is an advantage in terms of public health. The manufacturing process does not cause environmental pollution problems, which are becoming increasingly essential to avoid, and this is another of the aspects that differentiate these formulations from those belonging to the state of the art. Moreover, the process for manufacturing these new formulations has technical as well as economic advantages.

It is also desirable to avoid the initial lag time in the release of drug from controlled release composition hence the process for preparation of pharmaceutical composition of the present invention is described which dispenses an initial loading dose as an immediate release form and rest of the dose is released slowly over a period of time ensuring dosage administration once every 24 hours.

The prior art discloses that oxcarbazepine forms an oxidation product, diketoiminodibenzyl; 10,11-dihydro-5H-dibenzo[b,f] azepine-10,11-dione. Formation of this oxidation product leads to discoloration of oxcarbazepine.

We have designed the formulation in the present invention so that total impurities in the compostions are less than or equal to 2% by weight. The product also maintains other characteristics relating to the amount of oxcarbazepine, dissolution and total impurities for a period of validity of 3 years.

### SUMMARY OF THE INVENTION

It is an object of this invention to provide a novel oral solid pharmaceutical composition for oxcarbazepine in the form of a bilayer system that allows an immediate delivery of oxcarbazepine followed by a controlled release of oxcarbazepine from specialized matrix forming layer wherein the total amount of oxcarbazepine impurities in the composition is less than or equal to about 2% by weight.

The present invention is directed to an improved and more economical method for the stable and convenient treatment of epilepsy. Also the present invention is directed to a method for preparing a bioavailable controlled release 24-hour formulation for antiepileptic drug such as oxcarbazepine.

It is another object of the invention to provide a simple process of manufacturing for solid pharmaceutical composition for oxcarbazepine in the form of bilayer system.

It is also an object of the invention to provide a dosage form that can deliver the substantially aqueous insoluble drug oxcarbazepine at a controlled and beneficial known rate over time.

It is a further object of the present invention to provide the process for the preparation of such a composition, which releases an active agent from one layer instantaneously due to rapid disintegration of the layer in the fluid of the environment of use.

It is yet another object of the present invention to develop the process of preparation of bilayer system for delivering oxcarbazepine that maintains therapeutically active concentrations of oxcarbazepine with once a day administration thus leading to better patient compliance.

To these ends, the present invention consists in the provision of Bilayer oxcarbazepine tablets comprising:
a) a first or immediate release layer containing excipients including a diluent, a binder, a disintegrant, a lubricant and an oxcarbazepine or its salts or its polymorphs intended for immediate delivery;
b) a second or controlled release layer including an oxcarbazepine for controlled drug delivery, a matrix forming gelling agent, a wetting agent, a binder, hydrophilic solutes, and a lubricant.

When the Bilayer oxcarbazepine tablets, which on oral ingestion, come into contact with gastric fluid, the first layer disintegrates rapidly releasing the active ingredient instantaneously, the second layer considerably swells and gels in presence of fluid of the environment resulting release the active agent from second layer in a controlled manner.

The Bilayer oxcarbazepine tablets display and maintains the characteristics relating to the amount of oxcarbazepine, dissolution and total impurities suitable for a period of validity of 3 years, and the total amount of oxcarbazepine impurities is less than or equal to about 2% by weight.

The release of oxcarbazepine starts within 60 seconds and about 50% of the total drug contained in the tablet is released within one hour, and the remaining amount is released slowly up to a period of 12 hour duration.

The oxcarbazepine is delivered to maintain therapeutically active concentrations of oxcarbazepine with once a day administration whereby to lead to better patient compliance.

The total amount of oxcarbazepine present in the bilayer tablet varies between 100 mg and 1200 mg, and preferably between 150 mg and 600 mg.

The oxcarbazepine is present in the immediate release layer in the range of 20% to 80% of the active agent by weight based on the total weight of immediate release layer, and preferably of from 30% to 60% of the active agent by weight based on the total weight of immediate release layer.

The ratio of oxcarbazepine in immediate release layer to that in controlled release layer is in the range of 0.5:1 to about 1:15, and preferably in the range of from 0.5: to about 1:15.

The solid pharmaceutical tablet is coated and the layers have different colors for differentiation of the layers.

The disintegrating agent used in the immediate release layer is selected from the group consisting of starch, sodium starch glycolate, pregelatinished starch, crosslinked poly vinyl pyrrolidone, cross linked carboxy methyl cellulose, ion exchange resin, citric acid, tartaric acid, and sodium bicarbonate.

The immediate release layer includes crosslinked poly vinyl pyrrolidone in an amount ranging from about 0.25% to 5%, and preferably 0.5 to 3.0% crosslinked poly vinyl pyrrolidone and is about 2% by weight based on the total weight of immediate release layer.

The lubricant used in immediate and controlled release layer is selected from the group consisting of calcium stearate, magnesium stearate, sodium lauryl sulphate, light mineral oil, polyethylene glycol, stearic acid, talc, magnesium lauryl sulphate, sodium oleate, sodium acylate, silica derivatives, and sterotex, and wherein the magnesium stearate is present in an amount of 0.5% to 3.5%.

The diluent used is selected from the group consisting of maize starch, lactose, dicalcium phosphate, microcrystalline cellulose, and wherein said microcrystalline cellulose varies from 20% to 60%.

The controlled release layer includes oxcarbazepine, a matrix forming gelling agent, a wetting agent, binder, hydrophilic solutes, a lubricant, a coloring agent, and an antioxidant.

The oxcarbazepine is present in the range of 20% to 80%, preferably from 30% to 60% and most preferably 50% of the active agent by weight based on the total weight of the controlled release layer.

The binder is selected from the group consisting of cellulose ethers, methyl cellulose, ethyl cellulose, hydroxyl propyl cellulose, and hydroxypropyl methyl cellulose, and are present in an amount by weight of about 0.5% to about 15%, based on the total weight of controlled release layer, and in one preferred embodiment wherein the hydroxypropyl methyl cellulose is present in an amount of 5% to 6% by weight based on the total weight of controlled release layer.

The concentration of the matrix forming gelling agent is in the range of from about 5% to about 20.0% and preferably from 6% to 8% by weight based on the total weight of the controlled release layer.

The matrix forming gelling agent is selected from the group consisting of hydroxypropyl methylcellulose, methylcellulose, hydroxypropyl cellulose, hydroxyethyl cellulose, carbomer, carboxy methylcellulose, gum tragacanth, gum acacia, guar gum, pectin, modified starch derivatives, xanthan gum, locusta bean gum, and sodium alginate, and in one preferred embodiment the matrix forming gelling agent is a combination of two hydroxyethyl cellulose.

The combination of hydroxyethyl cellulose 250H and hydroxyethyl cellulose 250 L and the weight ratio of the 250H to 250 L is about 1:4 to 4:1, and preferably about 1:3 to about 3:1.

The hydrophilic solutes are selected from the group consisting of mannitol, sorbitol, galactilok, inositol, xylitol, glucose, altrose, xylulose, tagatose, sorbose, psicose, hamamalose, allose, their corresponding ketoses and deoxy forms, sedoheptulose, maltose, lactose, sucrose, cellobiose, isomaltose, dextrates and mixtures thereof.

The hydrophilic solutes are mannitol and dextrates and each is present in an amount from 10% to about 40% and preferably present in an amount of 12% to 25% of weight of the controlled release layer.

The ratio of mannitol to dextrates is from about 1:5 to about 5:1 and preferably from about 1:4 to about 4:1 and most preferably 1:1.

The wetting agent is sodium lauryl sulfate and is present in amount varying from 0.25 to 2% and preferably 0.5 to 1% based on the total weight of the controlled release layer.

The Bilayer oxcarbazepine tablets are film coated.

The present invention is also concerned with a process for the preparation of Bilayer oxcarbazepine tablets by wet granulation comprising the following steps:

Preparation of Immediate release granule I includes Sifting of Oxcarbazepine, diluent, disintegrant through 40# sieve and mixing in a suitable mixer fir 15 minutes; granulating the blend with water in fluidized bed granulator; drying of granules at 45-50° C. till LOD is between 2-3% w/w in fluidized bed drier; sifting of dried granules through 16# sieve; and lubricating the granules with lubricant, glidant (presifted through 40#) by mechanical mixing; and

Preparation of controlled release Granules II includes sifting of Oxcarbazepine, crystal habit modifier, matrix forming gelling agent, osmotically effective solutes, wetting agent through 30# sieve and mixed by mechanical means in an area with a controlled temperature and humidity; granulating the blend using water and drying of granules at 45-50° C. in fluidized bed drier; and then

Compression follows, in which Granules I and II are then compressed on a Manesty bilayer tablet compression machine.

Other features, advantages and objectives of this invention and its preferred embodiments will become apparent from the detailed description and accompanying claims, which follow.

### BRIEF DESCRIPTION OF THE FIGURES

FIG. 1 is a graph illustrating dissolution, and indicates accelerated stability study of Dissolution of Bilayer Oxycarbazepine tablets 600 mg, with the ordinate illustrating percent release and the abscissa time in hours, illustrating three time periods, one month, three months, and six months.

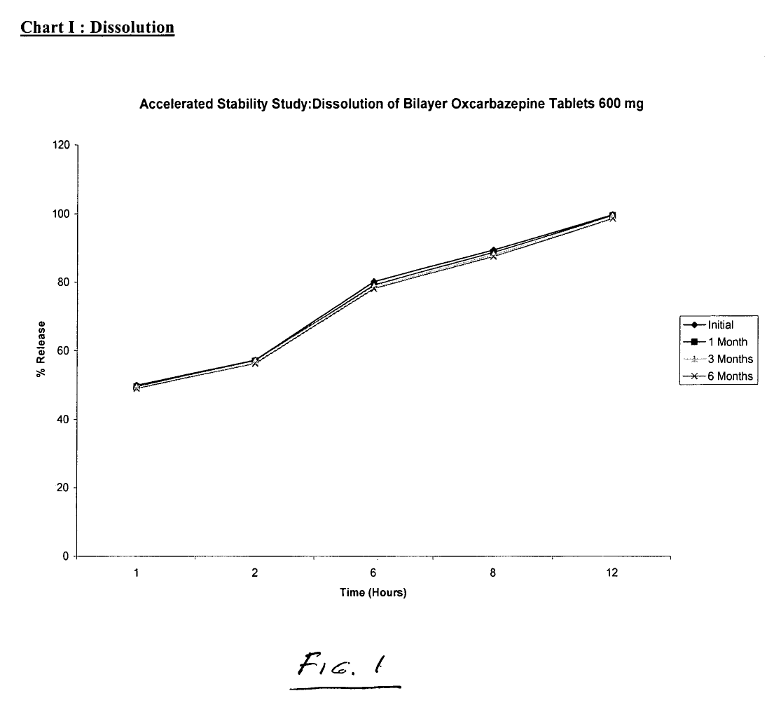

FIG. 2 is an Assay graph, also illustrating an accelerated stability study, and indicates the Assay of Bilayer Oxycarbazepine tablets and the abscissa illustrates the time period in months as in FIG. 1, and the ordinate indicating the percent assay.

### DETAILED DESCRIPTION OF THE FIGURES

In order to test the stability of the finished products in their packaged form, batches of oxcarbazepine bilayer tablets were packed in polyamide/aluminium/PVC blisters sealed with aluminium foil and aluminium strips. The blisters/aluminium strips were incubated in climatic chambers at 25.degree. C. and 60% relative humidity (atmospheric conditions) and 40.degree. C. and 75% relative humidity (accelerated conditions). The following parameters were periodically determined: assay, dissolution and level of impurities.

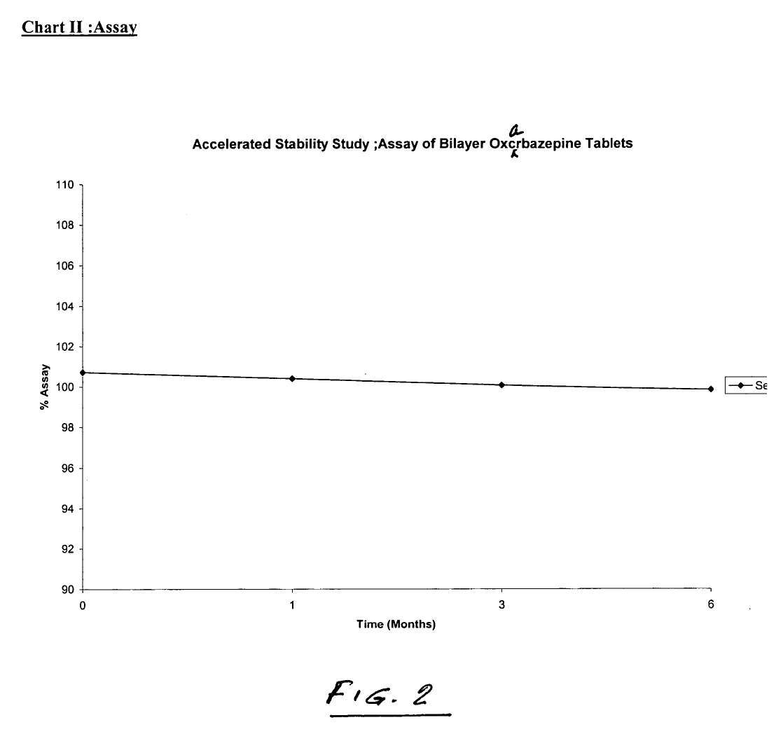

As noted heretofore, FIG. 2 shows a graph of the assay, dissolution results obtained for oxcarbazepine tablets in blisters and kept at 40.degree. C. and 75% relative humidity (accelerated conditions) for 6 months. As can be seen, the oxcarbazepine content is never lower than 90%, which is the minimum amount generally admissible for the amount of active ingredient in pharmaceutical forms during the period of validity. Also as regards the dissolution test, the values were determined at various time intervals over a period of 12 hours. It was found that within 1 hour, the entire amount of oxcarbazepine from immediate release layer was released and the entire amount oxcarbazepine from the controlled release layer was released slowly over a period of 12 hours. As can be seen, the dissolution at the end of 12 hours is never lower than 75%, which is the minimum amount generally admissible for the amount of active ingredient in pharmaceutical forms during the period of validity.

It was also found that total impurities of oxcarbazepine were less than 2% during the period of validity.

The values obtained for each of the parameters studied are presented in detail in Tables I and II.

TABLE I
Oxcarbazepine Bialyered Tablets 600 mg
STABILITY DATA

| | | | |
|---|---|---|---|
| Batch No | RD002 | Packing | Blister strip |
| Storage | 25 ± 2° C. & RH 60 ± 5% | | |
| Shelf Life | 3 Years | | |

Period in months

| | Initial | | |
|---|---|---|---|
| Description | Bilayer capsule | 3 | 6 |
| Dissolution (%) | shaped tablets | Satisfactory | Satisfactory |
| 1 hour | 49% | 49% | 49% |
| 2 hours | 57% | 57% | 57% |
| 6 hours | 80% | 79% | 79% |
| 8 hours | 89% | 88% | 88% |
| 12 hours | 99% | 99% | 99% |
| Related substances | 0.2 | 0.3 | 0.3 |
| (total impurities %) | | | |
| Assay (%) of | 100.7% | 100.5% | 100.2% |
| Oxcarbazepine | | | |

TABLE II
Oxcarbazepine Bialyered Tablets 600 mg
STABILITY DATA

| | | | |
|---|---|---|---|
| Batch No | RD002 | Packing | Blister strip |
| Storage | 40 ± 2° C. & RH 75 ± 5% | | |
| Shelf Life | 3 Years | | |

Period in months

| | | 1 | 3 | 6 |
|---|---|---|---|---|
| Description | Initial | Bilayer capsule | Satis- | Satis- |
| Dissolution (%) | Satisfactory | shaped tablets | factory | factory |
| 1 hour | 49% | 49% | 49% | 49% |
| 2 hours | 57% | 57% | 56% | 56% |
| 6 hours | 80% | 79% | 78% | 78% |
| 8 hours | 89% | 88% | 88% | 87% |
| 12 hours | 99% | 99% | 99% | 98% |
| Related substances | 0.2 | 0.2 | 0.3 | 0.5 |
| (total impurities %) | | | | |
| Assay (%) of | 100.7% | 100.4% | 100.0% | 99.8% |
| Oxcarbazepine | | | | |

### DETAILED DESCRIPTION OF THE INVENTION

The present invention describes a novel oral solid pharmaceutical composition for oxcarbazepine in the form of bilayer system that allows immediate delivery of oxcarbazepine followed by controlled release of oxcarbazepine from a specialized matrix forming layer.

The present invention provides solid pharmaceutical composition for oral administration containing two layers comprising:

a) a first or immediate release layer containing excipients and oxcarbazepine intended for immediate delivery; and

b) a second or controlled release layer that includes oxcarbazepine for controlled drug delivery, a matrix forming gelling agent, wetting agent, binder, hydrophilic solutes, lubricant, coloring agent.

As noted heretofore, the prior art discloses that oxcarbazepine forms an oxidation product, diketoiminodibenzyl; 10,11-dihydro-5H-dibenzo[b,f] azepine-10,11-dione. Formation of this oxidation product leads to discoloration of oxcarbazepine. Other impurities in the product are carbamazepine, methoxy carbamazepine, oxy minostilben.

We have designed the formulation in the present invention so that all impurities in the product are less than or equal to 2% by weight. The product also maintains other characteristics relating to the amount of oxcarbazepine, dissolution and total impurities for a period of validity of at least 3 years.

Release of oxcarbazepine starts within 60 seconds and about 50% of the total oxcarbazepine is released within one hour. The remaining amount is released slowly up to a period of 12 hours and thus facilitates once a day administration of oxcarbazepine.

The process for preparation of said composition of the present invention discloses a composition which on oral ingestion, comes into contact with gastric fluid, the first layer disintegrates rapidly releasing the active ingredient instantaneously, the second layer considerably swells and gels in the presence of fluid of the environment resulting in the release of the active agent from the second layer in a controlled manner.

The immediate release layer comprises oxcarbazepine, diluent, disintegrant, binder, lubricant, colour, antioxidant. Oxcarbazepine is present in immediate release layer in the range of 20% to 80%, preferably from 30% to 60% and most preferably about 50% of the active agent by weight based on the total weight of immediate release layer.

The present invention provides for a process for the preparation of a composition of the preferred embodiment as described by present invention in which the preferred ratio of the active agent in the immediate layer to that in the controlled release layer is in the range of 0.5:1 to about 1:15, more preferably from about 0.5:1 to about 1:5 and most preferably 1:1.

The disintegrating agent used in the immediate release layer can be selected from the group consisting of starch, sodium starch glycolate, pregelatinised starch, crosslinked poly vinyl pyrrolidone, cross linked carboxy methyl cellulose, ion exchange resin, the most preferred being crosslinked poly vinyl pyrrolidone. Crosslinked poly vinyl pyrrolidone helps in rapid disintegration of the first layer as the system comes into contact with the fluid of the environment thus releasing the active agent instantaneously. Cross linked poly vinyl pyrrolidone is present in an amount ranging from about 0.25% to 5%, more preferably 0.5 to 3.0% and most preferably is about 2% by weight based on the total weight of immediate release layer.

The diluent used is selected from maize starch, lactose, dicalcium phosphate, microcrystalline cellulose, and the most preferred diluent is microcrystalline cellulose. The amount of microcrystalline cellulose may vary from 15% to 80% and most preferably from 20% to 60%.

The lubricant used in immediate and controlled release layer is selected from calcium stearate, magnesium stearate, sodium lauryl sulphate, light mineral oil, polyethylene glycol, stearic acid, talc, magnesium lauryl sulphate, sodium oleate, sodium acylate, silica derivatives, sterotex, preferably magnesium stearate. Magnesium stearate is present in an amount ranging from about 0.5% to 5%, preferably up to 3.5% and most preferably is about 1%.

Oxcarbazepine is present in the controlled release layer in the range of from 20% to 80%, preferably from 30% to 60% and most preferably about 50% of the active agent by weight based on the total weight of controlled release layer.

The controlled release layer comprises oxcarbazepine, a matrix forming gelling agent, wetting agent, binder, hydrophilic solutes, lubricant, coloring agent.

Particularly suitable binding agents are cellulose ethers e.g. methyl cellulose or ethyl cellulose, hydroxypropyl methyl cellulose The dosage form of this invention, when it contains crystal habit modifiers such as hydroxypropyl methyl cellulose, contains them in a preferred amount by weight of about 0.5 to about 15%, preferably about 1% to about 14%, more preferably 4% to about 7%, and most preferably about 5% to 6% based on the amount of active agent.

The present invention provides the process for preparation of composition of the preferred embodiment where the second controlled release layer contains one or more matrix forming gelling agents selected from group consisting of hydroxypropyl methylcellulose, methylcellulose, hydroxyl ethyl cellulose, hydroxypropyl cellulose, carbomer, carboxy methylcellulose, gum tragacanth, gum acacia, guar gum, pectin, modified starch derivatives, xanthan gum, locusta bean gum, sodium alginate, the most preferred being hydroxy ethylcellulose which on contact with gastric fluid swells and forming matrix structure and also release oxcarbazepine in a controlled manner.

A combination of hydroxymethyl cellulose with hydroxyethyl cellulose is suitable. Also suitable is a combination of hydroxyethyl cellulose 250H and hydroxyethyl cellulose 250 L. In these designations, the “250” indicates the degree of substitution and the “L” and “H” refer to the viscosity and thereby the molecular weight. With particular reference to this combination, the weight ratio of the 250H to 250 L is preferably about 1:4 to 4:1, more preferably about 1:3 to about 3:1, still more preferably about 1:2 to about 2:1, most preferably about 2:1.

The concentration of the matrix forming gelling agent is from about 5% to about 20% and the more preferred being 6% to 15% and the most preferred being 8% by weight based on the total weight of the controlled release layer.

The controlled release layer of the present embodiment comprises osmotically effective solutes such as mannitol, sorbitol, galactilol, inositol, xylitol, glucose, altrose, xylulose, tagatose, sorbose, psicose, hamamalose, allose, their corresponding ketoses and deoxy forms, sedoheptulose, maltose, lactose, sucrose, cellobiose, isomaltose, and mixtures thereof, especially dextrates.

Preferably mannitol, dextrate is used. The dosage form of the present invention comprises preferably about 10% to about 40%, more preferably about 12% to about 25% of each of mannitol and dextrate weight of the controlled release layer.

The ratio of mannitol to dextrate is from about 1:5 to about 5:1, preferably about 1:4 to about 4:1, most preferably 1:1.

Oxcarbazepine is practically insoluble in water. A wetting agent is added to improve solubility of oxcarbazepine. Anionic, cationic or non ionic surfactants are preferred wetting agents It is selected from sodium, potassium or magnesium n-dodecylsulfate, n-tetradecylsulfate, n-hexadecylsulfate or n-octadecylsulfate, sodium, potassium or magnesium n-dodecyloxyethyl sulfate, n-tetradecyloxyethyl sulfate, n-hexadecyloxyethyl sulfate or n-octadecyloxyethyl sulfate; sodium, potassium or magnesium n-dodecanesulfonate, sorbitan monolaurate, sorbitan tristerate or triolate, polyethylene glycol fatty acid ester such as polyethylene glycol 400 sterate, polyethylene glycol 2000 sterate, ethylene oxide/propylene oxide block polymers of the Pluronics.®. (BWC) or Synperonic.®. (ICI) type, polyglycerol-fatty acid ester, glyceryl-fatty acid esters Most preferable is sodium lauryl sulfate, and it is preferably present in amount varying from 0.25 to 2%, more preferably from about 0.5%-1% based on the total weight of the controlled release layer.

The dosage form of the present invention may contain a coloring agent. The coloring agent may be selected from any colorant used in pharmaceuticals which is approved by the FDA.

The dosage form of the present invention may contain an antioxidant. The antioxidant may be selected from hydroxycarboxylic acids, such as tartaric acid, citric acid and gluconic acid, and pharmaceutically acceptable salts thereof, aminocarboxylic acids such as iminodiacetic acid, N-methyliminodiacetic acid, nitrolotriacetic acid, edetic acid (ethylenediamine tetraacetic acid), diethylenetriaminepentaacetic acid, 1,2-diaminocyclohexanetetraacetic acid or N-hydroxylethylenediaminetriacetic acid, and pharmaceutically acceptable salts thereof such as the alkali and alkaline earth salts,e.g., edetate calcium disodium, edetate disodium, edetate trisodium, and edetate tetrasodium. Edetate disodium is the preferred pharmaceutically acceptable antioxidants.

It may be film coated to protect it from moisture. The coating polymer is selected from a group of hydroxypropyl methylcellulose, hydroxypropyl cellulose, polyvinyl pyrrolidone, polyvinyl alcohol or methacrylic acid polymer, the most preferred being hydroxypropyl methylcellulose.

The present invention provides the process for preparation of composition of the preferred embodiment is as described below.

The granules for immediate and controlled release can be prepared either by wet granulation, dry granulation, slugging and compaction. The most preferred process being wet granulation.

Wet Granulation:

In the wet granulation process and technique, oxcarbazepine and the ingredients comprising the immediate release layer are individually passed through a 40 mesh screen and then all ingredients except lubricant are thoroughly blended in a mixer. Purified water is used as a granulating fluid. Purified water is slowly added to the drug blend with continual mixing in planetary mixer. Water is added until a wet blend is produced, which wet mass then is sieved through # 0.5 inch in multimill. The granules are dried at 45-50° C. till LOD is between 2-3% w/w. in fluidized bed drier. The dry granules are sized then through a 16 mesh screen. Next, a lubricant, preferably magnesium stearate is sifted through a 40 mesh screen and added to the dried granules. The granules are then blended for a period of 1 to 15 minutes.

Using the above procedure, lubricated granules for the controlled release layer is prepared. Then lubricated granules of both, i.e. immediate release and controlled release are compressed on a bilayer tablet compression machine.

Dry Granulation

Another manufacturing process that can be used for providing the compartment-forming composition comprises blending the powdered ingredients in a planetary mixer. The blend is then compressed into slugs. Slugs are milled into granules and sifted through a 16# sieve. A lubricant such as magnesium stearate is added to granules and mixed for 10-15 minutes.

Using the above procedure lubricated granules for the controlled release layer is prepared. Then lubricated granules of both, i.e. immediate release layers and controlled release are compressed on a bilayer tablet compression machine.

The invention will be more fully understood from the following examples. These examples are to be constructed as illustrative of the invention and not limitative thereof:

### EXAMPLE 1

Example 1 discloses the wet granulation process for preparation of bilayer tablet according to the present invention.

| | % w/w of total wt. Of |
|---|---|
| 1. Ingredients of Immediate layer | immediate release layer |
| Oxcarbazepine | 50.0 |
| Microcrystalline cellulose | 46.0 |
| Crospovidone | 2.0 |
| Anhydrous Colloidal Silica | 0.2 |
| Magnesium stearate | 1.8 |

| 1. Ingredients of Controlled release | % w/w of total wt. Of |
|---|---|
| layer | controlled release layer |
| Oxcarbazepine | 50.0 |
| Hydroxypropyl methylcellulose | 7.0 |
| Hydroxy ethyl cellulose 250 L | 2.5 |
| Hydroxy ethyl cellulose 250 H | 5.0 |
| Mannitol | 16.9 |
| Dextrate | 16.9 |
| Sodium lauryl sulphate | 0.7 |
| Magnesium stearate | 1.0 |

The process for preparation of composition of Example 1 is as follows:

Preparation of Immediate release granule I:

a) Oxcarbazepine, microcrystalline cellulose, crospovidone are sifted through 40# sieve and mixed in a suitable mixer for 15 minutes.

b) The blend is granulated with water by mechanical means.

c) Granules are dried at 45-50° C. till LOD is between 2-3% w/w in fluidized bed drier.

d) Dried granules are mechanically sifted through 16# sieve.

e) The granules are lubricated with magnesium stearate, colloidal anhydrous silica (presifted through 40#) by mechanical mixing.

Preparation of controlled release Granules II:

Oxcarbazepine, Hydroxypropyl methyl cellulose, Hydroxyethyl cellulose 250 L, Hydroxyethyl cellulose 250H, mannitol, dextrates, sodium lauryl sulphate are sifted through 30# sieve and mixed by mechanical means in a area with a controlled temperature and humidity.

The blend is granulated using water and granules are dried at 45-50° C.

The dried granules are sifted through 16# sieve and lubricated with magnesium stearate (presifted through 40# sieve).

Compression:

Granules I and II are compressed on core coat (e.g. Dry cota/Press cota) tablet compression machine.

### EXAMPLE 2

Example 2 discloses a process for preparation by dry granulation according to the present invention

Dry Mix

| 1. Ingredients of Immediate release | % w/w of total wt. Of |
|---|---|
| layer | immediate release layer |
| Oxcarbazepine | 50.0 |
| Microcrystalline cellulose (Avicel) | 46.0 |
| Crospovidone | 1.5 |
| Anhydrous Colloidal Silica | 0.5 |
| Magnesium stearate | 2.0 |

| 2. Ingredients of Controlled release | % w/w of total wt. Of |
|---|---|
| layer | controlled release layer |
| Oxcarbazepine | 47.0 |
| Microcrystalline cellulose (Avicel) | 21.5 |
| Hydroxypropyl methylcellulose | 4.0 |
| Hydroxy ethyl cellulose 250 L | 2.0 |
| Hydroxy ethyl cellulose 250 H | 4.0 |
| Mannitol | 10.0 |
| Dextrate | 10.0 |
| Sodium lauryl sulphate | 0.5 |
| Magnesium stearate | 1.0 |

The process for preparation of composition of Example 2 is as follows:

Preparation of Immediate release granule 0:

a) Oxcarbazepine, microcrystalline cellulose, crospovidone, colloidal anhydrous silica magnesium stearate, talc are sifted through a 40# sieve and mixed in a suitable mixer for 15 minutes.

b) The blend is compressed into slugs by mechanical means.

c) Slugs are then milled through multimill.

d) Dried granules are mechanically sifted through a 16# sieve.

e) The granules are lubricated with a mixture of magnesium stearate, (presifted through a 40#) by mechanical mixing.

f) Lubricated granules are then compressed on a compression machine.

Preparation of controlled release Granules II:

a) Oxcarbazepine, Hydroxypropyl methyl cellulose, microcrystalline cellulose, Hydroxyethyl cellulose 250 L, Hydroxyethyl cellulose 250H, mannitol, dextrates, sodium lauryl sulphate are sifted through 30# sieve and mixed by mechanical means in a area with a controlled temperature and humidity.

b) The blend is compressed into slugs. Slugs are then milled to yield granules.

c) The granules are sifted through 16# sieve and lubricated with magnesium stearate (presifted through 40# sieve).

Compression:

Granules I and II are compressed on core coat (e.g. Dry cota/Press cota) tablet compression machine.

### EXAMPLE 3

Example 3 discloses film coated bilayer tablet according to the present invention. Process of core tablets is same as that in Example 2.

| 1. Ingredients of Immediate release | % w/w of total wt. Of |
|---|---|
| layer | immediate release layer |
| Oxcarbazepine | 52.0 |
| Microcrystalline cellulose | 44.57 |
| Crospovidone | 1.3 |
| Colloidal Silicone Dioxide | 1.0 |
| Magnesium stearate | 1.13 |

| 2. Ingredients of Controlled release | % w/w of total wt. Of |
|---|---|
| layer | controlled release layer |
| Oxcarbazepine | 53.5 |
| Hydroxypropyl methylcellulose | 6.3 |
| Methocel K 4M | 8.03 |
| Lactose | 30.37 |
| Sodium lauryl sulphate | 0.8 |
| Magnesium stearate | 1.0 |

| 3. Coating | % w/w of total weight of tablet |
|---|---|
| Hydroxypropyl methyl cellulose | 2.0 |
| Glycerin | 0.8 |
| Colour | 0.1 |

Coating:

Appropriate quantity of hydroxypropyl methylcellulose, glycerin, colour is dissolved in purified water under stirring to get clear solution. The solution is strained through 100# & used for film coating of tablets.

It is to be understood that the examples and embodiments described hereinabove are for the purposes of providing a description of the present invention by way of examples and are not to be viewed as limiting the present invention in any way. Modifications that may be made to that described in above examples by those of ordinary skill in the art is also contemplated by the present invention and is to be included within the scope of the invention as disclosed.

With optimized dosage forms—final dosage forms it will be shown below that the products obtained, as well as obeying analytic specifications when analyzed immediately after manufacture, maintain their stability during a period of time appropriate for sale on the pharmaceutical market for therapeutic purposes.

Control of the Finished Product

Parameters analyzed:

1) Assay

2) Dissolution

3) Total impurities

Stability Tests

To test the stability of the pharmaceutical dosage forms, stability tests were performed on batches of oxcarbazepine bilayer tablets manufactured according to the present invention. The tablets were packed in a) blisters of polyamide/aluminium/PVC sealed with aluminium foil b) aluminium strips. These blisters/strips are maintained at room temperature (real time) and also in an incubator at 40.degree. C. and 75% relative humidity, and certain parameters were periodically determined, such as assay, dissolution total impurities. The results obtained for one batch of oxcarbazepine bilayer tablets are presented in the aforenoted Tables I and II.

The World Health Organisation considers that accelerated stability studies using incubation conditions of a temperature of 40/degrees/C and 75% relative humidity for 3 months in temperate and subtropical climates and 6 months in hot climates for products that are intended for the global market is a good indicator of the stability of a pharmaceutical dosage form. (WHO/PHARM/94.565).

While there has been shown and described the various details of the invention, it will be obvious to those skilled in the art that various changes and modifications may be made that come within the scope of the teachings of this invention.

1. Bilayer oxcarbazepine tablets comprising:
a) a first or immediate release layer containing excipients a diluent, a binder, a disintegrant, a lubricant and an oxcarbazepine or its salts or its polymorphs intended for immediate delivery;
b) a second or controlled release layer including an oxcarbazepine for controlled drug delivery, a matrix forming gelling agent, a wetting agent, a binder, hydrophilic solutes, and a lubricant.

2. Bilayer oxcarbazepine tablets as in claim 1, which on oral ingestion, comes in contact with gastric fluid, the first layer disintegrates rapidly releasing the active ingredient instantaneously, the second layer considerably swells and gels in presence of fluid of the environment resulting release the active agent from second layer in a controlled manner.

3. Bilayer oxcarbazepine tablets as in claim 1, wherein the tablet displays and maintains the characteristics relating to the amount of oxcarbazepine, dissolution and total impurities suitable for a period of validity of 3 years, the total amount of oxcarbazepine impurities in less than or equal to about 2% by weight.

4. Bilayer oxcarbazepine tablets as in claim 1, wherein release of oxcarbazepine starts within 60 seconds and about 50% of the total drug contained in the tablet is released within one hour, and the remaining amount if released slowly up to a period of 12 hour duration.

5. Bilayer oxcarbazepine tablets as in claim 1, for delivering oxcarbazepine to maintain therapeutically active concentrations of oxcarbazepine with once a day administration whereby to lead to better patient compliance.

6. Bilayer oxcarbazepine tablets as in claim 1, wherein total amount of oxcarbazepine present in the bilayer tablet varies between 100 mg and 1200 mg.

7. Bilayer oxcarbazepine tablets as in claim 1, wherein total amount of oxcarbazepine present in the bilayer tablets varies between 150 mg and 600 mg.

8. Bilayer oxcarbazepine tablets as in claim 1, where the Oxcarbazepine is present in the immediate release layer in the range of 20% to 80% of the active agent by weight based on the total weight of immediate release layer.

9. Bilayer oxcarbazepine tablets as claimed in claim 1, wherein Oxcarbazepine is present in immediate release layer in the range of from 30% to 60% of the active agent by weight based on the total weight of immediate release layer.

10. Bilayer oxcarbazepine tablets as claimed in claim 1, where the ratio of oxcarbazepine in immediate release layer to that in controlled release layer is in the range of from 0.5:1 to about 1:15.

11. Bilayer oxcarbazepine tablets as claimed in claim 1, where the ratio of oxcarbazepine in immediate release layer to that in controlled release layer is in the range of from 0.5: to about 1:15.

12. Bilayer oxcarbazepine tablets as claimed in claim 1 for use in the treatment of convulsive states.

13. Bilayer oxcarbazepine tablets as claimed in claim 1, wherein the solid pharmaceutical tablet is coated and the layers have different colors for differentiation of the layers.

14. Bilayer oxcarbazepine tablets are claimed in claim 1, wherein the disintegrating agent used in the immediate release layer is selected from the group consisting of starch, sodium starch glycolate, pregelatinished starch, crosslinked poly vinyl pyrrolidone, cross linked carboxy methyl cellulose, ion exchange resin, citric acid, tartaric acid, and sodium bicarbonate.

15. Bilayer oxcarbazepine tablets as claimed in claim 14 wherein the immediate release layer includes crosslinked poly vinyl pyrrolidone in an amount ranging from about 0.25% to 5%.

16. Bilayer oxcarbazepine tablets as claimed in claim 16, wherein the immediate release layer includes 0.5 to 3.0% crosslinked poly vinyl pyrrolidone and is about 2% by weight based on the total weight of immediate release layer.

17. Bilayer oxcarbazepine tablets as claimed in claim 1, wherein the lubricant used in immediate and controlled release layer is selected from the group consisting of calcium stearate, magnesium stearate, sodium lauryl sulphate, light mineral oil, polyethylene glycol, stearic acid, talc, magnesium lauryl sulphate, sodium oleate, sodium acylate, silica derivatives, and sterotex, and wherein the magnesium stearate is present in an amount of 0.5% to 3.5%.

18. Bilayer oxcarbazepine tablets as in claim 1, wherein the diluent used is selected from the group consisting of maize starch, lactose, dicalcium phosphate, microcrystalline cellulose, and wherein said microcrystalline cellulose varies from 20% to 60%.

19. Bilayer oxcarbazepine tablets as claimed in claim 1, wherein controlled release layer includes oxcarbazepine, a matrix forming gelling agent, a wetting agent, binder, hydrophilic solutes, a lubricant, a coloring agent, and an antioxidant.

20. Bilayer oxcarbazepine tablets as claimed in claim 19, wherein the oxcarbazepine is present in the range of 20% to 80% of the active agent by weight based on the total weight of the controlled release layer.

21. Bilayer oxcarbazepine tablets as claimed in claim 19, wherein the oxcarbazepine is present in the amount of 50% of the active agent by weight based on the total weight of the controlled release layer.

22. Bilayer oxcarbazepine tablets as claimed in claim 19, wherein the binder is selected from the group consisting of cellulose ethers, methyl cellulose, ethyl cellulose, hydroxyl propyl cellulose, and hydroxypropyl methyl cellulose, and are present in an amount by weight of about 0.5% to about 15%, based on the total weight of controlled release layer.

23. Bilayer oxcarbazepine tablets as claimed in claim 22, wherein hydroxypropyl methyl cellulose is present in an amount of 5% to 6% by weight based on the total weight of controlled release layer.

24. Bilayer oxcarbazepine tablets are claimed in claim 19, wherein the concentration of matrix forming gelling agent is in the range of from about 5% to about 20.0% by weight based on the total weight of the controlled release layer.

25. Bilayer oxcarbazepine tablets as claimed in claim 24, wherein the concentration of matrix forming gelling agent is from 6% to 8% by weight based on the total weight of the controlled release layer.

26. Bilayer oxcarbazepine tablets as claimed in claim 19, wherein the matrix forming gelling agent is selected from the group consisting of hydroxypropyl methylcellulose, methylcellulose, hydroxypropyl cellulose, hydroxyethyl cellulose, carbomer, carboxy methylcellulose, gum tragacanth, gum acacia, guar gum, pectin, modified starch derivatives, xanthan gum, locusta bean gum, and sodium alginate.

27. Bilayer oxcarbazepine tablets as claimed in claim 19, wherein the matrix forming gelling agent is a combination of two hydroxyethyl cellulose.

28. Bilayer oxcarbazepine tablets as claimed in claim 27, wherein the combination of hydroxyethyl cellulose 250H and hydroxyethyl cellulose 250 L and the weight ratio of the 250H to 250 L is about 1:4 to 4:1.

29. Bilayer oxcarbazepine tablets as claimed in claim 27, wherein the combination of hydroxyethyl cellulose 250H and hydroxyethyl cellulose 250 L and the weight ratio of the 250H to 250 L is about 1:3 to about 3:1.

30. Bilayer oxcarbazepine tablets as claimed in claim 19, wherein the hydrophilic solutes are selected from the group consisting of mannitol, sorbitol, galactilok, inositol, xylitol, glucose, altrose, xylulose, tagatose, sorbose, psicose, hamamalose, allose, their corresponding ketoses and deoxy forms, sedoheptulose, maltose, lactose, sucrose, cellobiose, isomaltose, dextrates and mixtures thereof.

31. Bilayer oxcarbazepine tablets as claimed in claim 30, wherein the hydrophilic solutes are mannitol and dextrates and each is present in an amount from 10% to about 40% of weight of the controlled release layer.

32. Bilayer oxcarbazepine tablets as claimed in claim 30, wherein the hydrophilic solutes are mannitol and dextrates and each is present in an amount of 12% to 25% of the weight of the controlled release layer.

33. Bilayer oxcarbazepine tablets as claimed in claim 30, wherein the ratio of mannitol to dextrates is from about 1:5 to about 5:1.

34. Bilayer oxcarbazepine tablets as claimed in claim 30, wherein the ratio of mannitol to dextrates is from about 1:4 to about 4:1.

35. Bilayer oxcarbazepine tablets as claimed in claim 20, wherein the wetting agent is sodium lauryl sulfate and is present in amount varying from 0.25 to 2% based on the total weight of the controlled release layer.

36. Bilayer oxcarbazepine tablets as claimed in claim 1, wherein the tablet is film coated.

37. A process for the preparation of bilayer oxcarbazepine tablets by wet granulation comprising the following steps:
I. Preparation of Immediate release granule I:
Sifting of Oxcarbazepine, diluent, disintegrant through 40# sieve and mixing in a suitable mixer for 15 minutes;
Granulating the blend with water in fluidized bed granulator;
Drying of granules at 45-50° C. till LOD is between 2-3% w/w in fluidized bed drier;
Sifting of dried granules through 16# sieve; and
Lubricating the granules with lubricant, glidant (presifted through 40#) by mechanical mixing;
II. Preparation of controlled release Granules II:
Sifting of Oxcarbazepine, crystal habit modifier, matrix forming gelling agent, osmotically effective solutes, wetting agent through 30# sieve and mixed by mechanical means in an area with a controlled temperature and humidity;
Granulating the blend using water and drying of granules at 45-50° C. in fluidized bed drier; and
Sifting the dried granules through a 16# sieve and lubricating the granules with a lubricant (presifted through 40# sieve);
III. Compression:
Granules I and II are then compressed on a Manesty bilayer tablet compression machine.
